(19) United States
(12) Patent Application Publication
(10) Pub. No.: US 2021/0022827 A1
(43) Pub. Date: Jan. 28, 2021

(54) DEVICES AND METHODS OF TISSUE VISUALIZATION FOR USE IN LAPAROSCOPIC, ROBOT-ASSISTED LAPAROSCOPIC, AND OPEN PROCEDURES

(71) Applicants: The Board Of Regents for Oklahoma State University, Stillwater, OK (US); The Board of Regents of the University of Oklahoma, Norman, OK (US)

(72) Inventors: DAQING PIAO, Stillwater, OK (US); SANJAY PATEL, OKLAHOMA CITY, OK (US)

(21) Appl. No.: 16/317,855

(22) PCT Filed: Jul. 17, 2017
(86) PCT No.: PCT/US2017/042367

§ 371 Date: Jan. 15, 2019

Related U.S. Application Data
(60) Provisional application No. 62/362,862, filed on Jul. 15, 2016.

(51) Int. Cl. A61B 90/00; A61B 34/37

(52) U.S. Cl. CPC A61B 90/361; A61B 2034/301; A61B 34/37

(57) ABSTRACT

Taught herein is a new contact-based optical imaging technology, en-face differential optical topography (en-face DOT), which performs real-time visualization of subsurface tissue heterogeneity within a depth up to 3 mm and over a 9.5 mm diameter FOV with a modest mm-level lateral resolution. An embodiment of the probe fits in a 12 mm port and houses at its maximum 128 cop-per-coated 750 μm fibers that form radially alternating illumination (70 fibers) and detection (58 fibers) channels. By simultaneously illuminating the 70 source channels of the laparoscopic probe that is in contact with a scattering medium and concurrently measuring the light diffusely propagated to the 58 detector channels, the presence of near-surface optical heterogeneities can be resolved in an en-face 9.5 mm field-of-view in real-time. Visualization of subsurface margin of strong attenuation contrast at a depth up to 3 mm is demonstrated at one wavelength at a frame rate of 1.3 Hz.

### CROSS REFERENCE TO RELATED APPLICATIONS

This application claims the benefit of U.S. Provisional Patent Application serial number 62/362,862 filed on Jul. 15, 2016, and incorporates said provisional application by reference into this document as if fully set out at this point.

### TECHNICAL FIELD

This disclosure relates generally to medical visualization and, in more particular, to systems and methods associated with imaging tools of the sort used in minimally-invasive procedures.

### BACKGROUND

Laparoscopic and robot-assisted procedures increasingly have become the preferred approaches for patients and surgeons, specifically in urologic oncological surgery. When compared to open surgery, procedures done laparoscopically are impaired by the difficulty of obtaining timely intraoperative pathology consultation, stereotactically confined field-of-view and decreased (pure laparoscopic surgery), and nonexistent (robot-assisted laparoscopic surgery) tactile sensation. With these challenges, surgeons operating laparoscopically often have to rely upon subjective visual cues to guide excision of tumors (i.e. adequate resection of renal tumor in partial nephrectomy (PN)) and avoiding tumor violation or iatrogenic injury to critical adjacent tissues (i.e. identifying the prostate capsule and preserving peri-prostatic nerve tissues during radical prostatectomy (RP)).

Among the laparoscopic imaging tools available for intraoperative tissue assessment, drop-in ultrasound is particularly useful for localizing tumor depth; however, the tumor depth evaluated in the transverse ultrasound view does not directly indicate the tumor margin over the lateral field-of-view (FOV) whereupon the resection is performed. Laparoscopic wide-field imaging of Firefly fluorescence has shown to enhance the outcome of PN. Wide-field imaging of the surface fluorescence is done in a lateral FOV that is ideal for direct navigation guidance; yet, as the image mainly reflects superficial fluorescence emission, tissue heterogeneity beneath the surface due to invasive lesions could be missed.

A laparoscopic imaging tool that allows intraoperative tissue evaluation including assessment of the tumor margin in PN or identification of the prostate capsule amid peripheral nerve tissues in RP is desirable to probe tissue contrast (due to absorption, scattering, and fluorescence, etc.) at a few millimeters depth, visualize over the lateral view for resection guidance, have a non-microscopic FOV adequate for rapid survey of the resection site, and form the image in real-time. Probing light diffusely propagated through tissue provides sub-surface sensitivity as with diffuse reflectance imaging or spatial frequency domain imaging, but the image formation generally involves intense computation that may be costly to the intraoperative time-frame. Projecting these modalities laparoscopically to sample subsurface tissue heterogeneity over a non-microscopic FOV for rapid site-survey has been challenging.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

According to an embodiment, there is provided a laparoscopic optical imaging technology, en-face differential optical topography (en-face DOT), which performs real-time visualization of subsurface tissue heterogeneity within a depth of a few millimeters and provides a two dimensional view over the width and length of the instrument tip which might be, in some embodiments, 9.5 mm diameter instrument-tip FOV. This en-face DOT device combines well-defined depth-sampling of separate source and detector channels and spatial resolution rendered by the maximum-density fiber channels.

An embodiment of en-face DOT uses a two-dimensional fiber source/detector array as is common to diffuse reflectance imaging or tomography. But an embodiment of the en-face DOT invention operates differently in at least three aspects: (1) the source/detector fiber-array is set at a higher density; and, (2) the signals originating from all source channels are acquired simultaneously, i.e., there is no multiplexing of the source channels; and, (3) the output is a two dimensional image representative of the near subsurface of the object imaged.

The all-channels-ON mode of one embodiment might resemble using an imaging-fiber (1 mm or less FOV when in contact with tissue), but there are fundamental differences. An imaging-fiber has fully coherently ordered fiber channels, and the individual μms-size fiber is used for both illumination and light-detection thereby the imaging essentially samples the surface. In comparison, an embodiment of the instant en-face DOT probe has semi-coherently ordered fiber channels. Each, for example, 100 s-μm size fiber acts as either the source-channel or detector-channel, but not both, to render depth-sampling that is unavailable to an image-fiber. This high-density semi-coherent fiber-array combined with all-sources-ON operation directly maps in real-time the lateral distributions of optical sources of contrast, at a 1-2 mm sampling depth depending upon the diameter of the individual fiber, over an en-face FOV sized by the cross-sectional dimension of the applicator probe, with a lateral resolution related to the size of the individual fiber. Various embodiments can be configured to probe subsurface spectral or fluorescence heterogeneity and using non-contact applicator.

According to one particular example there is provided a laparoscopic applicator probe that houses 128 optical fibers that comprise intermixed, e.g., radially alternating, illumination (70 fibers) and detection (58 fibers) channels. By simultaneously illuminating the 70 source channels and concurrently measuring the light diffusely propagated to the 58 detector channels, near-surface optical heterogeneities can be resolved in an en-face 9.5 mm FOV in real-time. Visualization of subsurface margin of strong attenuation contrast at a depth up to 3 mm is possible at one wavelength at a frame rate of 1.3 Hz. An embodiment of the laparoscopic en-face DOT may be extended to probe near-surface spectral and fluorescence heterogeneities, and with non-contact probe for scaling the FOV and imaging depth, for the potential of intraoperative tissue margin assessment.

Note that, for purposes of the instant disclosure, that when the term “laparoscopic” is used herein, that term should be broadly construed to include endoscopic, open procedures, etc. In other words, although the specific examples provided in the discussion that follow might be couched in terms of laparoscopic procedures, this invention is not to be limited to that particular surgical procedure but, instead, should be considered to be applicable to sort of surgery-related imaging.

In one embodiment, an imaging device for imaging a tissue is provided that comprises: a light source; a plurality of source fibers, each of said plurality of source fibers having a source fiber first end positionable to be in optical communication with the light source and an emitting end; a plurality of detector fibers, each of said plurality of detector fibers having an imaging end positionable to be in optical communication with a light sensor and a detector end positioned to detect light from said plurality of source emitting ends that falls on the tissue; and, a probe having an open upper end and an open terminus, wherein said upper end receives said plurality of source fibers and said plurality of detector fibers therein and guides said source and receiver fibers to said open terminus where said source and detector fibers emerge intermixed in a two-dimensional array of source fiber emitting ends and detector fiber ends within said probe terminus.

In another embodiment, there is provided an imaging device, comprising: a first light source; a plurality of first source fibers, each of said plurality of first source fibers having a first source fiber first end positionable to be in optical communication with the light source and a first source emitting end; a second light source different from said first light source; a plurality of second source fibers, each of said plurality of second source fibers having a second source fiber end positionable to be in optical communication with said second light source and a second source emitting end; a plurality of detector fibers, each of said plurality of detector fibers having an imaging end positionable to be in optical communication with a light sensor and a detecting end; and, a probe having an open upper end and an open terminus, wherein said upper end receives said plurality of first source fibers, said plurality of second source fibers, and said plurality of detector fibers therein and guides said first source fibers, said second source fibers, and said detector fibers to said open terminus where said first source fibers, said second source fibers, and said detector fibers emerge intermixed to form a two dimensional array of first source emitting ends, second source emitting ends, and detector ends within said probe terminus.

In a further embodiment, there is provided a method of imaging a tissue, wherein is provided a probe and a plurality of source fibers and a plurality of detector fibers, each of said plurality of source fibers having a source end and an emitter end, and each of said detector fibers having a detector end and an imaging end, wherein said probe encases said source fibers and said detector fibers and terminates in plurality of intermixed said source ends and said detector ends that form a two-dimensional array at an end of said probe, comprising the steps of: activing said light source; exposing each of said source ends to said light source; directing said probe toward the tissue; while said light source is activated collecting light from each of said imaging ends of said detector fibers, thereby obtaining at least one light intensity value for each of said detector fibers; associating each of said detector fiber ends with a position within said probe terminus; and, using said position within said probe terminus associated with each of said detector fiber ends and said at least one light intensity value for each of said detector fibers to form a two-dimensional image of said tissue.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1 contains a system level schematic illustration of an embodiment.

FIG. 2 contains a detailed view of the terminus of the probe according to one embodiment.

FIG. 3 contains an operating logic suitable for use with an embodiment.

FIG. 4 contains a schematic representation of how the detector fibers are converted into a two-dimensional image.

FIG. 5 contains some examples of how well anomalies at different depths might be imaged. FIG. 5A illustrates an embodiment where the probe is in direct contact with the tissue. FIG. 5B illustrates an embodiment where the probe is positioned above the tissue. FIG. 5C contains an illustration of an embodiment which utilizes an array of source/detector fibers in direct contact with the tissue that is to be imaged. FIG. 5D contains an illustration of the embodiment of FIG. 5C where the probe is not in direct contact with the tissue.

FIG. 6 illustrates a method and apparatus for improving the spatial resolution of an embodiment. In FIG. 6A, contains an example of a normal resolution embodiment. FIG. 6B contains a schematic illustration of an approach that would produce an image of enhanced spatial resolution. FIG. 6C contains a variation of FIG. 6B with a different combination of sources and detectors in operation. FIG. 6D contains still another variation of FIG. 6B with a different combination of sources and detectors in operation.

FIG. 7 contains a schematic illustration of a system embodiment suitable for use with the approach of FIG. 6.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

According to an embodiment, there is provided a laparoscopic contact-based optical imaging technology, en-face differential optical topography (en-face DOT, hereinafter), which performs real-time visualization of subsurface tissue heterogeneity within a depth of a few millimeters and over, for example, a 9.5 mm diameter instrument-tip FOV with mm-level lateral resolution. The en-face DOT device combines well-defined depth-sampling of simultaneously activated source and detector channels with 2-D spatial resolution provided by a collection of high-density fiber channels combined with image reconstruction by, for example, interpolation between detectors to form a real-time 2-D image. Note that, for purposes of the instant disclosure, when the term “fiber” is used herein it is intended to refer to an optical fiber or a light guide as those terms are known and understood in the art. A fiber might be, for example, comprised of a core of glass or plastic surrounded by a cladding material and, in some cases, a protective cover or sheath might optionally enclose the core and cladding. Also, in some cases extrapolation might be used to widen the field of view somewhat. As such, when the word “interpolate” is used herein that should be broadly construed to also include instances where extrapolation is used in addition to interpolation.

By way of explanation, an en-face DOT embodiment uses a high density two-dimensional fiber-array of light sources and light receivers/detectors that operate simultaneously to acquire a two-dimensional image. This embodiment of the en-face DOT technology has a fiber-array in which the source fibers emit light contemporaneously and the detector fibers are operational while the light is being emitted. Each detector fiber is read separately and the multiple detectors are then combined (e.g., by interpolation) to create a two dimensional image of the surface and/or the near surface of the subject tissue.

The all-channels-ON mode differs from traditional imaging-fiber (1 mm or less FOV when in contact with tissue), but there is at least one fundamental difference. An imaging-fiber has fully coherently ordered fiber channels, and the individual μms-size fiber is used for both illumination and light-detection so that the imaging essentially samples the surface. In comparison, the en-face DOT probe has semi-coherently ordered fiber channels., e.g., the individual 100 s-μm size fiber acts as either the source-channel or detector-channel, but not both, to render depth-sampling that is unavailable to an image-fiber. This unique high-density semi-coherent fiber-array combined with all-sources-ON operation directly (thus real-time) maps the lateral distributions of optical sources of contrast, e.g., at a 1-2 mm sampling depth, over an en-face FOV sized by the applicator probe, with a lateral resolution largely determined by the individual fiber size. Although some embodiments utilize probe light attenuation at single-wavelength, the apparatus and method can be configured to probe subsurface spectral or fluorescence heterogeneity.

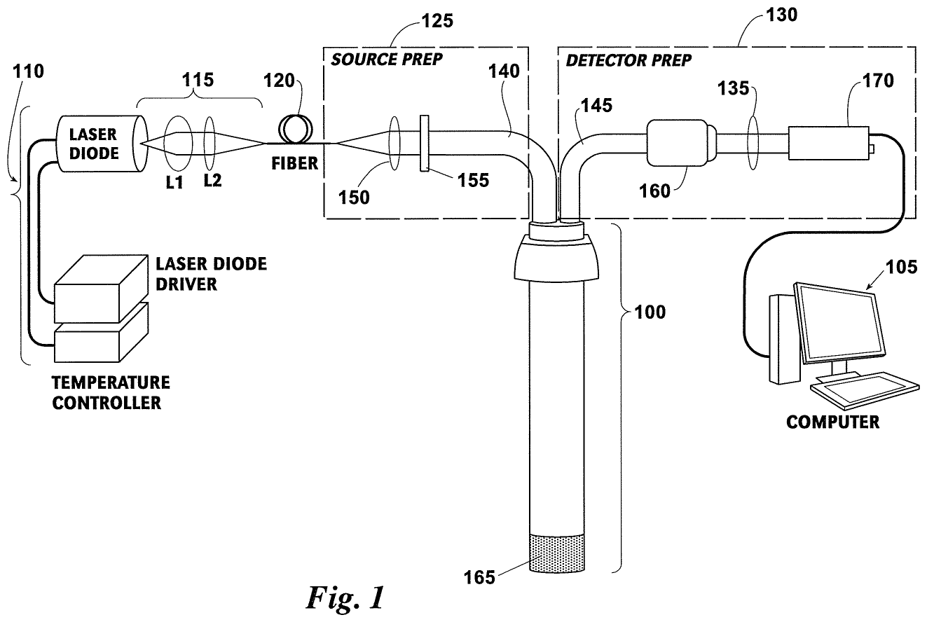

Turning first to FIG. 1 which contains an exemplary embodiment, in that figure the probe 100 contains two bundles of fiber optic fibers: a source bundle 140 that is comprised of a number of source fibers and a detector bundle 145 that is comprised of a number of detector fibers. In this particular example, the source bundle 140 is in optical communication with a laser diode driver and laser diode combination 110 that generate the light that is emitted from the terminus 165 of the probe 100 when the light source is activated. Of course, a laser light source is only one example of the sort of light source that might be used. Other light sources such as LED lights might also be used. In one embodiment, a 785 nm laser diode can be used as a light source.

Continuing with the example of FIG. 1, in this embodiment one or more convex or concave lenses 115 (e.g., convex lenses L1 and L2) may optionally be used to prepare the generated light for entry into a fiber transmission line 120 which conveys the light between the light source 110 and the source bundle 140.

Additionally, it may be useful to prepare the transmitted light 125 by passing it through a convex or concave lens complex 150 which collimates the light from the transmission line 120 and disperses it on a diffuser 155 before the light enters the source bundle 140. One reason that this particular approach might be useful is that is homogenizes the light from the source so that each of the termini of the source fibers in the bundle 140 is equally illuminated.

The light in the source bundle 140 travels through the probe 100 and is emitted through its terminus 165 which houses the termini of the source fibers in the bundle 140. In some embodiments there might be 70 source fibers. If the source and receiver fibers are 100 s-μm, the result is an image with a field of view of 9.5 mm in diameter that consists of 128 circles each representing a tissue area of 750 μm in diameter. Clearly, the number of source and detector fibers used with a probe of this diameter might be varied depending on the size the diameter of the source and detector fibers. Additionally, there is no requirement that the source and detector fibers be the same diameter. As a specific example given the dimensions of the current example, the exemplary count of 128 fibers might be varied to as much as plus 500% or minus 75% of that count in some applications. If the probe is sized differently and/or has a non-cylindrical interior, a different number of fibers might be used. Using the example provided, that might mean that there could be as many as 350 source fibers and 300 or so detector fibers. At the opposite extreme, it is desirable that there not be fewer than about 50 source fibers and 40 detector fibers. Of course, the numerical examples given are not rules but are merely suggested counts that might be useful in some circumstances.

Light from the terminus of the probe 165 is projected onto the subject tissue as is described in greater details below. That light then illuminates the termini of the plurality of fibers that comprise the detector bundle 145, the fibers of which are intermixed with the termini of the source bundle 140. In some embodiments, there might be 58 detector fibers. The light that is collected by the termini of the fibers in the detector bundle 145 is conducted upward and through the probe and, in the example of FIG. 1, prepared 130 for use by the computer 105 by passing the light from the detector bundle through a microscope objective lens 160 and a convex or concave relay lens 135 before impinging on a light sensor 170 which might be a charge coupled device (“CCD”) of the sort found in a digital camera. That being said, all that is required is some sort of device that can sense light signals and convert those light signals to electrical impulses which can be used to determine the relative intensity of the light that falls on each location (e.g., pixel) of the device. Although a CCD is in many ways preferred, other devices that can actively sense light (e.g., CMOS chips, photodiode arrays, multi-channel PMTs) might be used instead. So, for purposes of the instant disclosure, when the term “light sensor” is used herein, or in the claims that follow, that term should be broadly construed to include any such light detecting device including, without limitation, a CCD and a CMOS chip.

Light from each fiber in the detector bundle 145 is converted to optical intensity signals by the sensor 170 and transmitted (via wire or wirelessly) to a computing device (e.g., a laptop or desktop computer, a table computer, or any sort of conventional or unconventional computing device that contains a CPU and an accessible display device 175). The display device 175 will be configured to display processed signals from the sensor 170 as described below. Preferably the display device 175 will be operable in real-time to display signals from the terminus 165 of the probe as it moved on a subject's body. Of course, those of ordinary skill in the art will recognize that the CPU might be, for example, in a desktop or laptop personal computer. It could also be a tablet computer or a custom designed computing device. In short, any programmable device that can translate the electrical information from the CCD into information displayable on the display device 175 would be acceptable. Additionally, it is also possible that the CCD, CPU, and display might be incorporated into a single device, e.g., within the light sensor. In that case, the positioning of the detector fibers to faun an image (i.e., positioning the information from the detector fibers in image space) and display of that image would be handled within that single device.

By way of a specific example, in one embodiment a bundle of 70 source fibers is evenly illuminated by a laser diode (785 nm, 525B Laser Diode Driver and TED 200C Temperature Controller, Thorlabs Inc., Newton, N.J.) after collimating by a lens (C220TME-B, Thorlabs Inc., Newton, N.J.) and passing through a diffuser (10 deg 25 mm, Edmund Optics, Barrington, N.J.). The bundle of 58 detector fibers is imaged onto a camera (PointGrey GS3-U3-28S4M-C, 1928×1448 pixels) using a 4× microscope objective lens and a relay lens (100 mm focal length). When the probe is in contact with a 1% intralipid solution corresponding to a reduced scattering coefficient of 1 mm⁻¹ and an absorption coefficient of 0.0025 mm⁻¹ at 785 nm, one frame of raw image data is acquired in 8 ms at a camera gain of OdB, using the vendor-provided FlyCapture® 2.8 interface. The raw image data when processed off-line on a CORE i5 processor running on Windows® 7 for image formation takes approximately 78 ms. A streamlined interface developed in LabVIEW® incorporating MATLAB® scripts of image formation algorithm currently runs at 1.3 Hz.

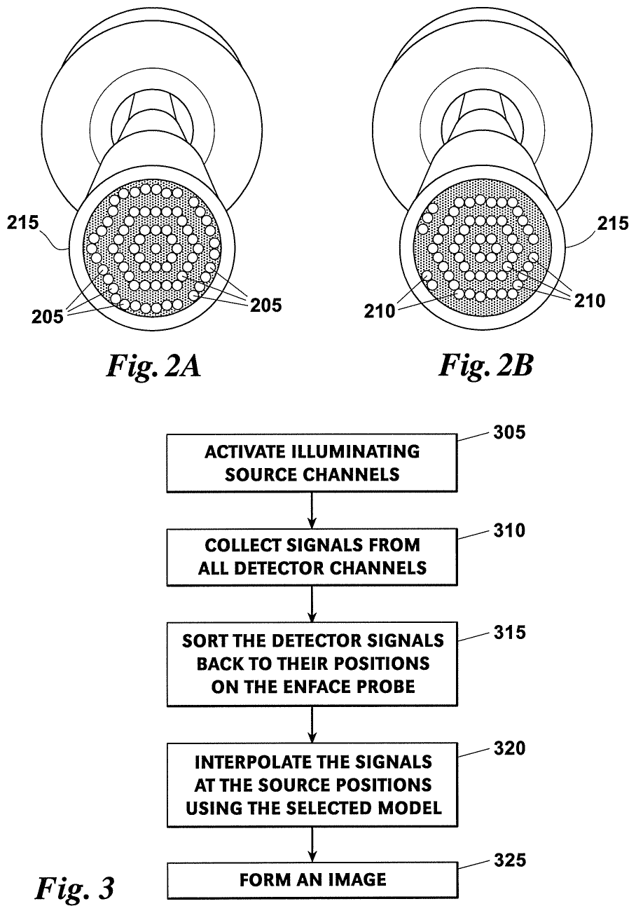

FIG. 2 contains a schematic representation of the end of the probe terminus 165 which illustrates one possible configuration of the termini of the fibers in the source bundle 140 and the detector bundle 145. In this particular example, the fiber termini 205 correspond to fibers from the source bundle 140 and the fiber termini 210 correspond to fibers from the detector bundle 145. As can be seen, in the preferred arrangement the fibers 205 and 210 will be closely packed together in a regular arrangement so a high resolution image of the subject material can be obtained. In this particular example, the fibers 205 and 210 are arranged in alternating concentric circles around a central source fiber 205, with the odd circles corresponding to source fibers 205 and the even circles corresponding to detector fibers 210. Obviously, whether the sources and detectors are arranged in a circular, rectangular, octagonal, etc., arrangement is not an important aspect of the invention. All that is required is that the source and detector fibers be packed closely together and at least approximately alternating in some sense. The source/detector fibers need not necessarily be regularly spaced (e.g., in alternating rows, columns, circles, etc.) but instead could be randomly intermixed with each other so long as the detectors 210 are close enough together to provide a two-dimensional view of sufficient resolution.

According to one specific example, there are 70 source fibers 205 and 58 detector fibers 210, each 750 μm in diameter. That being said, there is no particular reason for the source and detector fibers to all be the same diameter and, in some cases, it might be useful to have one or the other larger in diameter relative to the other. Those of ordinary skill in the art will be readily able to devise a suitable arrangement depending on the particular application for which the embodiment is intended. In some variations, the fibers 205 and 210 will be copper coated.

Continuing with the previous example, an en-face DOT laparoscopic probe was prepared with a FOV of 9.5 mm in diameter, using a standard blunt-tip trocar fitting a 12 mm stability sleeve port. The optical tip of the bladeless trocar was carefully removed to open up the stainless steel stem for housing optical fibers. A total of 128 copper-coated 750 μm fibers (Oxford Electronics, IR600/660, core/cladding/coating 600/660/750 nm, 0.22 NA) were enclosed by the tip-removed stainless steel stem. These fibers form approximately 6 circles concentric to the approximate center of the probe occupied by a fiber. A total of 70 fibers forming the odd number of circles are used as the source channels. The remaining 58 fibers forming the even number of circles are used as the detector channels. Due to limitations in fabrication process, the fibers at the periphery of the probe may be less evenly distributed as compared with those in the inner part of the probe.

Continuing with the example of FIG. 2, in this particular case the 128 source and detector fibers were packing into a 9.5 mm stainless steel cylindrical tube 215 (e.g., a trocar stem) which was housed within a 12 mm trocar. Those of ordinary skill in the art will recognize that the diameter of the tube 215 is not a critical aspect of this embodiment and it could be smaller or larger in diameter, e.g., from 50 mm (e.g., 50 mm for use in open surgery) to 2 mm (e.g., 2 mm for use in endoscopic surgery). However, what is important is that the source 140 and detector 145 fibers be packed close enough together within the terminus of the tube 215 so that the subject tissue is uniformly illuminated and the information from the detector bundle can be used to construct a full two-dimensional image (based on, e.g., attenuation, absorption, fluorescence, reflection, etc.) of the subject tissue using only the light from the source bundle 145. Clearly, the particular dimensions of the probe and the fiber that will be useful in a particular case may need to be determined by trial and error and those of ordinary skill in the art will readily be able to determine same for a particular application.

Turning next to FIG. 3, this figure contains an operating logic for an imaging function that is suitable for use with an embodiment. As a first step (not shown), the probe will be positioned on the subject tissue that is to be imaged. In some embodiments, the probe tip will be placed in direct contact with the subject tissue as is discussed further below.

The light source will need to be activated (box 305), after which data collection can commence (box 310) via the detector fibers 210. The light detected via the fibers 210 will be transmitted back to some aspect of the detector apparatus (e.g., a CCD 170) where it will be converted into electronic signals that include a light intensity for each detector fiber 210. For purposes of specificity in the text that follows, the term “CCD” will be used in a generic sense to refer to any light sensitive surface that can convert illumination data into electronic data (e.g., via the photoelectric effect). That being said, those of ordinary skill in the art should note that this disclosure is not limited to this particular sort of device.

In some embodiments, the imaging function will continue by associating each detector fiber 210 with its position on the probe terminus. Since one goal is to assembly a two-dimensional image from the detectors, knowledge of each fiber's position on the end of the probe will be important. Because of, for example, manufacturing issues the detector fibers 210 might not be positioned exactly where they were intended to be. In that case, the best image will result where each fiber's location on the probe terminus has been measured exactly before the probe is put into production.

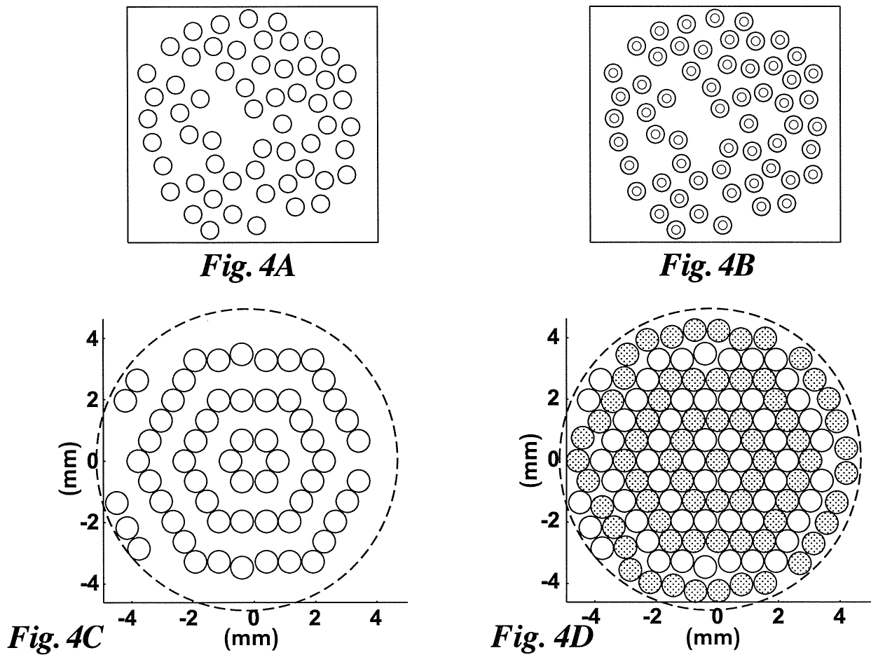

When light from the detector fibers arrive at the detection device 170 the fibers may randomly ordered so some association will need to be made between the fiber and its position within the probe so that each fiber may be properly located for use during image construction. The fibers at the tissue-imaging end are packed side by side, with the detector and source channels are interspaced. When the detector channels are combined into a bundle for transmission, their physical positions in the fiber-bundle 145 will not necessarily be the same as they are in the tissue-imaging end of the probe terminus. For example, although the detector fibers within the bundle might be radially interspersed with the source channels at the tissue-imaging end (e.g., as in FIG. 2), they will likely be randomly positioned in the bundle at the camera side due to, for example, the fabrication process. As a result, the exemplary raw image data as perceived by the CCD 170 or other detection device might contains image intensities that are essentially randomly positioned (FIG. 4A) in the viewing field. This configuration will need to be accounted for in order to provide a useful image of the subject tissue. Box 315.

This repositioning might be done in many ways, but one way that has proven to be useful in some embodiments is to prepare a table that identifies each light source as it appears in the coordinate system of the detection device and relates that image spot to a physical optical fiber location in the tissue end/terminus of the probe. Given that sort of information those of ordinary skill in the art will readily be able to associate the image intensity (or intensities) in the CCD/detection device 170 with a physical location in the probe terminus, i.e., position each of the detector fibers in the image space of the display device. Additionally, in some cases it might be useful to provide X and Y coordinates (relative to some arbitrary origin) for each detector fiber terminus in the probe. That information, in combination with the diameter of the fiber will be useful in the steps that follow.

Of course, the detector fibers do not completely cover the image but, instead, have designed gaps in them that are occupied in some embodiments by source fibers. Thus, in order to produce a two dimensional image it will be necessary to use the available light intensities to fill out a full image (box 320 of FIG. 3), e.g., via interpolation. FIGS. 4B, 4C, and 4D contain a schematic illustrate of one way this might be done. That being said, those of ordinary skill in the art will readily be able to devise alternative approaches.

According to one approach, the light intensity from each fiber in the detector bundle will likely illuminate multiple pixels on the surface of the CCD. In that case, an average of the pixel intensities will be calculated. In the example of FIG. 4B, the outer circle represents the total number pixels illuminated by each of the detector fibers and the inner circle represents the location on the display where the center of the fiber is located. The use of a weighted average or other measure of composite intensity could also be used (e.g., median, mode, etc.). In some embodiments, an empty (e.g., zero filled) display matrix will be prepared to receive the calculated intensities. In some embodiments, that matrix will be formed in memory in a configuration that reflects that of the terminus of the probe (e.g., circular) and will contain empty cells (e.g., memory locations) that correspond to the location of each detector fiber in the probe. Preferably, this matrix, once it has been filled, will contain the information that is used to formulate a complete image for viewing by the user via the display device 175.

As is generally suggested by FIGS. 4C and 4D, after each detector fiber (i.e., the clear circles in FIG. 4C) has been located within the display matrix and populated with the average intensity at that detector location, image intensity information for each of the locations in the probe which contains a light source will be generated, in effect turning the detectors into sources and the sources into detectors. As mentioned above, interpolation might be used to fill out the rest of the display matrix. In the example of FIG. 4D, locations that correspond to the sources (the stippled circles of this figure) have been filled in by interpolation so that the detector intensities, in combination with the interpolated sensor locations, provides a full two-dimensional image of the subject tissue. Whether a linear, spline, regression-based, or other interpolation algorithm is used is not critical to the operation of the instant invention and those of ordinary skill in the art will readily be able to select a method of interpolation that provides good results in a particular situation.

Finally, given the filled display matrix of FIG. 4D, an image can be displayed to the user on a display device. Of course, in order for that image to be of most use the process described above will need to be repeatedly performed in real time as the probe is moved. As a specific example, it is desirable to have the image updated at least as often as 30 times a second but the effective update rate can certainly be higher or lower depending on the needs of the user.

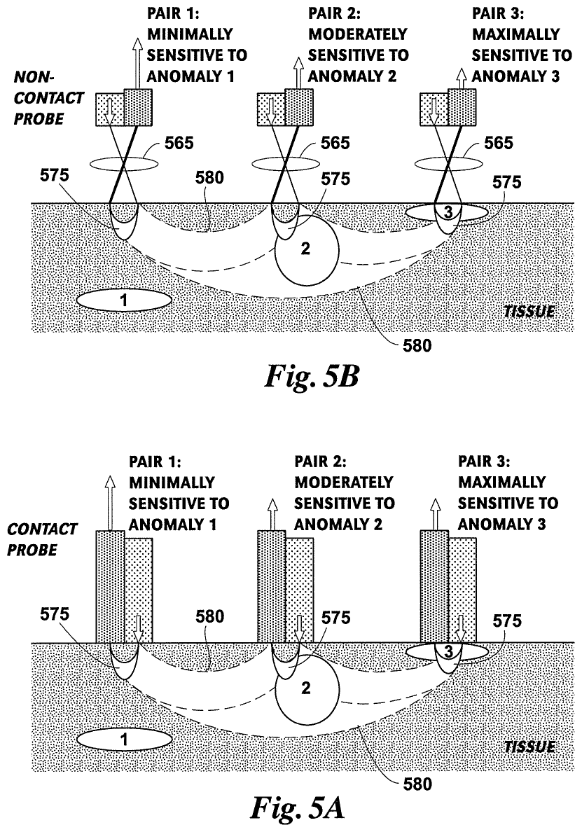

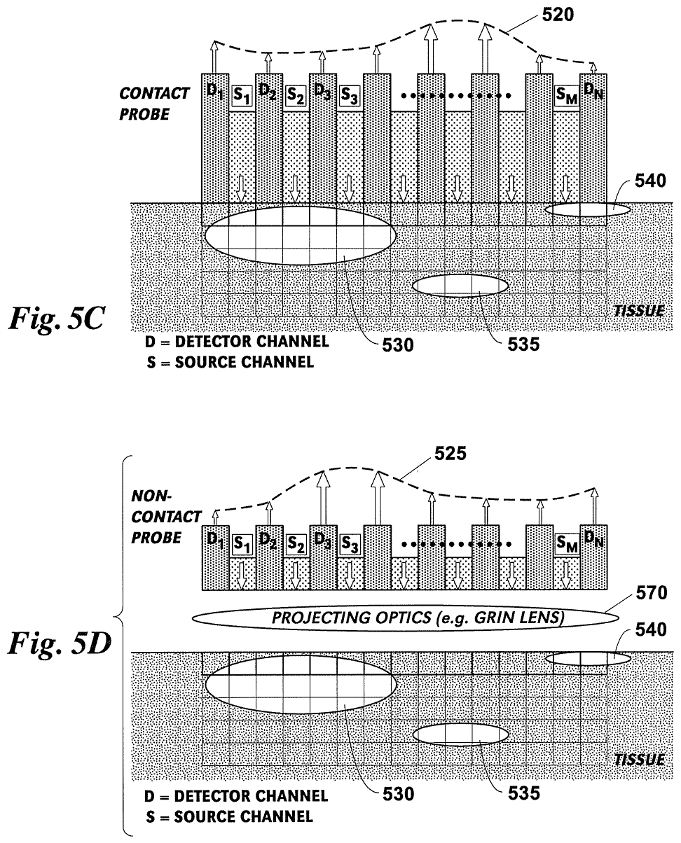

FIG. 5 contains some illustrations of how some embodiments of the instant disclosure might operate in practice to detect an anomaly that is situated below the surface of the subject tissue in two different situations: when the probe is in direct contact with the tissue that is to be imaged and when it is used to image the surface of the subject tissue without contacting it directly. In this figure, S₁, . . . , S_{M}, represent source channels and D₁, . . . , D_{M}, represent detector channels. As might be expected, the detectability of anomalies 505, 510, and 515, within otherwise relatively homogeneous tissue using an embodiment depends generally on the distance of the probe from the surface of the tissue that is being investigated, and the depth and extent of the anomaly.

As is indicated in FIG. 5A, when the probe is in direct contact with the surface of the tissue a relatively deep anomaly 1 will not be strongly detected, as might be expected. This is, at least in part, because the most direct light rays 575 from the closest source fiber to the nearest detector fiber in each source/detector pair (Pairs 1, 2, and 3), do not penetrate very deeply into the surface of the imaged tissue. Further, the light rays 580 from remote sources (e.g., Pair 1 and Pair 3 will be considered “remote” for this example) are too diffuse to provide much information about the deeper anomalies 1. The responsiveness of the detectors to an anomaly nearer the surface 2 is more pronounced and, of course, when the anomaly 3 is just be below the surface the detector will be maximally sensitive to it. In general, each detector fiber will be most sensitive to the anomaly present in the light path between the detector and its closest source. Note that for purposes of this disclosure, in the figures a downward pointing arrow corresponds to a light source that is directed from a source fiber toward the tissue and an upward arrow represents the light that is recovered by a detector fiber.

The situation where the probe is not in direct contact with the tissue (FIG. 5B) produces a similar result. If the probe is to be used remote from the surface of the subject tissue, it is preferred that some sort of lens 565 be used.

Turning next to FIGS. 5C and 5D, these figures indicate in a general way the responsiveness of an array of source/detector pairs (as opposed to spaced apart individual source/detector pairs). First with respect to FIG. 5C, this figure contains a schematic illustration of a multi-source and detector linear array that is in contact with the tissue that is to be imaged. Curve 520 gives a representation of a response light intensity curve for this array when used to detect anomalies 530, 535, and 540. As can be seen, this configuration is more responsive to the smaller and deeper anomaly 535 that to the shallower anomalies 530 and 540. However, and by way of comparison, FIG. 5D contains a response curve 525 to the same sorts of anomalies 530, 535, and 540, which is representative of a situation where the probe is not in contact with the tissue. Note that, in this case, the resulting image intensity is more responsive to the large and shallow anomaly 530 than to the deeper 535 and smaller 540 anomalies. In this particular case, it has proven to be useful to include a lens 570 (e.g., a gradient index or GRIN lens).

A 2D array of side-by-side source and detector fibers with alternative rows of source and detector channels resolves a shallow anomaly in an en-face view at a spatial resolution of one and two the fiber size in the source and detector directions, respectively.

Note that the probe configuration might be varied in different ways depending on the situation and what is desired to imaged. For example, the end of the probe might be adapted to house a single lens or optical element. Then, when operated in non-contact mode (e.g., at some distance from the subject tissue), the optics will be used to project the entire end profile of the fiber probe to the tissue surface.

As another example of a probe configuration when it is operated in non-contact mode, the probe terminus could be fitted with multi-element optics such as a micro-lens array that is aligned with the fiber channels. This will project each fiber channel to the tissue surface.

In still another embodiment of a probe that is suited for a non-contact application, micro-optics (e.g., a GRIN lens) could terminate each fiber channel to project the fiber channel to the tissue surface.

These are just a few examples of how a probe embodiment might be modified to operate differently in a non-contact mode.

In other variations the properties of the light source can be altered to highlight different aspects of the target. For example, the light source might be a single wavelength (e.g., λ₁) or multiple wavelengths (e.g., λ₁, λ₂, . . . , λ_{N}) which could be either discrete wavelengths or a broadband source. In the multi-wavelength case, the resulting image can be decomposed into its constituent frequencies according to methods well known to those of ordinary skill in the art, thereby making it possible to view the subject tissue under different illumination conditions.

In another variation, the light source might be a single wavelength (e.g., λ₁) but the

CCD could be pre-filtered to only accept light at a different frequency, say, λ₂. This could be useful if the intent was to study subsurface tissue via fluorescence. If λ₂<λ₁ then this would be an example of upper conversion. If λ₂>λ₁, that would be usually the situation if fluorescence were to be studied.

Another embodiment utilizes a broad band source with a known light frequency distribution (e.g., flat, weighted toward high frequencies or low frequencies, etc.). The resulting image is then analyzed to determine how the frequency distribution changes, e.g., as the probe is moved across the tissue or how the frequency distribution at one location differs from that at another location. This approach would make possible diffuse reflectance spectroscopy over the field over view of the probe.

In still another embodiment some of the source fibers might be illuminated with one light source and others of the source fibers illuminated with a different light source. As an example, one source might be a single frequency light source and the other source might be a broadband light source of known distribution. That would make it possible to form a single frequency image (via digital or optical filtering of the detector bundle) and/or perform a diffuse reflectance spectroscopy analysis as discussed previously.

In a further embodiment, the probe containing the bundles of source and detector fibers light additionally be equipped with an imaging fiber or fiber bundle which could be, for example, centrally located in the probe. That is, this variation includes a high resolution small field-of-view microscopy fiber or bundle surrounded, for example, by alternating rings of source and detector fibers as described previously. In this embodiment, the light source could be single frequency or broad band which would open up the possibility of, for example, fluorescence analysis or diffuse reflectance spectroscopy analysis coupled with a high magnification view of the subject tissue.

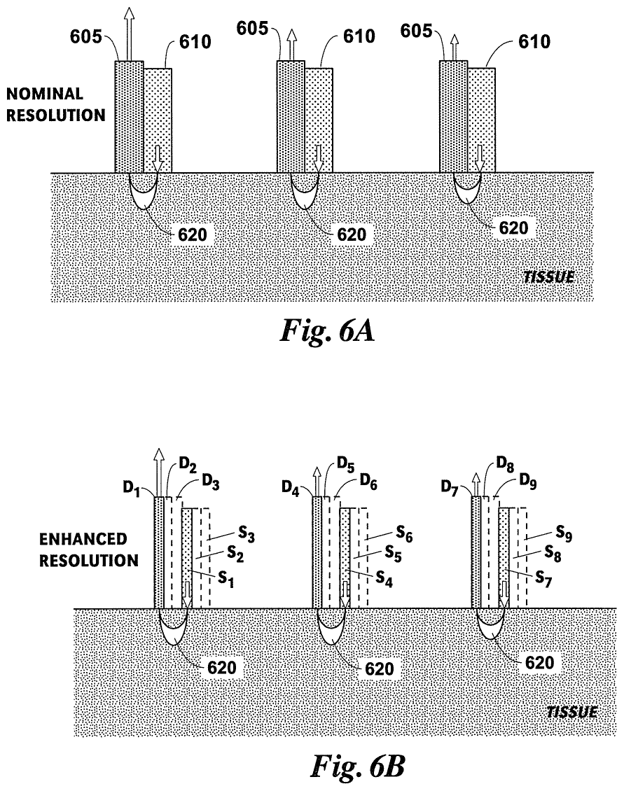

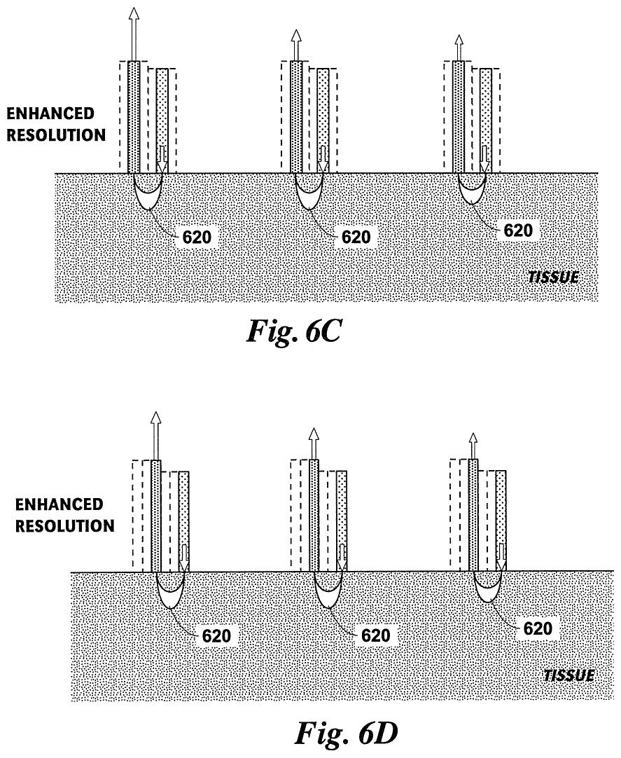

In another variation there is provided a system and apparatus generally as described above, but which features improved spatial resolution without compromising the effective sampling depth. FIG. 6 contains an example of such a variation. FIG. 6A contains a schematic illustration of the embodiment discussed above with sources 610 and detectors 605.

The source/detector pairs have been moved further apart that would normally be used for purposes of clarity. In this example, the source (“S”) and detector (“D”) fibers of diameter “d” are densely packed and alternated S/D/S/D/S/D/S/D. The arc 620 represents light from the source 610 that is conducted, reflected, dispersed, scattered, etc., by the tissue and is recovered by the detector fiber 605.

FIGS. 6B through 6D contain various views of an example scheme that would improve the spatial resolution without compromising the effective sampling depth. Note that although the examples in these figures are illustrated as having fiber channels arranged along a line, the extension to a two-dimensional array is straightforward. FIGS. 6B through 61) illustrate an embodiment which utilizes densely packed fibers of size d/3 which are grouped as three source fibers followed by three detector fibers, i.e., S1S2S3/D1D2D3/S4S5S6/D4D5D6 . . . in these figures. In operation, the group of fibers S1/D1/S4/D4 . . . is activated first (FIG. 6B), where activated means that the light that sources S1, S4, etc., is illuminated and the light returning through detectors, D1, D4, etc., is sensed. Next, as indicated in FIG. 6C, the group of fibers S2/D2/S5/D5 . . . is activated, followed by the group that consists of S3/D3/S6/D6 . . . (FIG. 6D). On activation of each group, the SD distance of d is the same as that in FIG. 6A, but the spatial resolution is 3-times better as compared with that example.

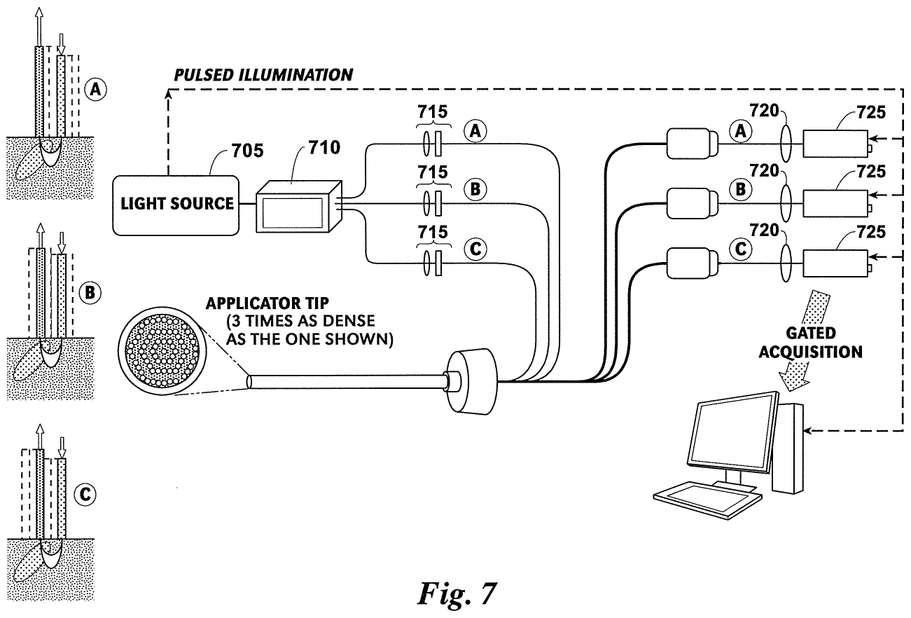

Note that variations shown in FIG. 6B-D are designed to improve the spatial resolution 3 fold as comparing with the embodiment in FIG. 6A. Clearly, this variation is not limited to improving the resolution 3 folds comparing to that in FIG. 6A. By implementing more steps than the 3-step shown in FIG. 6B-D, the spatial resolutions can be improved more than 3 folds comparing to that in FIG. 6A. FIG. 7 illustrates one approach to utilizing the technique of FIG. 6. The light source 705 in this particular example which might be, for example, a laser diode (“LD”) or an LED light source, will be fiber-switched 710 to three routes (A, B, and C) and homogenized 715 for illuminating three groups of source channels. The LD will be gated to operate at a sub-millisecond pulsed mode and synchronized with gated CCD acquisition, an approach that has been successfully applied in the past to intraoperative imaging of surface fluorescence for use under normal operating room lighting. For example, one embodiment uses a 750 nm LD that can be externally modulated and a LED at 785 nm for external modulation at a speed up to 20 KHz. Only 8 ms exposure time may be needed for the en-face OT at a continuous wattage source power of <1 mW and a CCD gain of 0. An exposure time less than 100 μs is expected to be achieved by pulsed operation of the source at approximately 10 mW power (−3 mW/cm² for a 2 cm projected beam diameter). The power density can be higher as the surgical field is imaged, not directly viewed, in laparoscopic procedures and at higher CCD gain. The use of a LD may be preferred instead of LED in some cases because of an additional laser speckle effect which may be present with the en-face OT applicator probe that can render another set of tissue scattering information. Each of three detection bundles A′, B′, and C′, in the example of FIG. 7 will be projected to a microscope objective 720, followed by band-pass filtering of the pulsed light, for raw data acquisition by a machine vision CCD camera of 90 Hz frame rate (time-sharing among 3 CCDs to allow >26 Hz frame rate).

For the high-density array, there is a total of M source positions and N detector positions (N=M+1 or M=N+1), the tissue length in contact with the array as marked by the horizontal solid arrow can be divided into (M+N) segments, each representing a column beneath a fiber. The complete set of signals acquired at the N detector positions and interpolated for M source positions thus qualitatively profiles the shallow tissue heterogeneity with a lateral resolution set by the fiber size. When a two-dimensional array with high fiber density is used as in (C), tissue anomaly at a depth interrogated by a side-by-side SD pair is mapped over the en-face plane. Spectral or fluorescence measurements can be readily achieved by using such a high-density fiber array to probe beneath-surface spectral heterogeneity or exogenous contrast. Note that, the source and detector channels as schematized in (C) alternates only along one direction, in contrary to a fully mixed and irregular source and detector channels in a square applicator probe of 2 cm×2 cm size for rapid data acquisition. What is demonstrate is real-time image formation using a circular laparoscopic probe within which the fibers form radially alternating positions for source and detector channels.

An algorithm for image formation is described below with reference to a particular example. Continuing with the example provided above, the 58 detector fibers within the bundle for coupling into the camera are randomly positioned due to the fabrication process. As a result, a raw image data acquired by the CCD or camera contains light intensities from the 58 detector fibers in a random arrangement. The circular region-of-interest (ROI) on the raw image data corresponding to each detector fiber-channel has an average radius of 60 pixels. The average intensity within a circular ROI of a radius of 20 pixels centered at each fiber position on the raw image data is calculated. A blank matrix of 1448×1928 identical in matrix size to the raw image is produced with the vertical dimension representing 9.5 mm—the diameter of the tissue-contacting fiber-probe. The average light intensity corresponding to each detector fiber position on the raw image as calculated is mapped onto every pixel of a circular ROI in the blank matrix corresponding to the actual position of the detector fiber on the probe. The pixel values for the ROI corresponding to each source fiber position of the 70 source fibers on the probe is assigned according to the method described in below.

Continuing with this specific example, denote ψₕₒₘₒ(\vec{r}ᵢ^{S}, \vec{r}ⱼ^{D}, λ) as the signal at a wavelength λ from a homogeneous medium when acquired at a detector position of \vec{r}ⱼ^{D} and illuminated at a single source position of rs. When all sources illuminate simultaneously, the detector at \vec{r}ⱼ^{D} measures

ψₕₒₘₒ(\vec{r}ⱼ^{D}, λ)=Σᵢψₕₒₘₒ(\vec{r}ᵢ^{S}, \vec{r}ⱼ^{D}, λ), j=[1, N].

When an anomaly is present and the sources illuminate simultaneously, the detector at \vec{r}ⱼ^{D} measures ψₕₑₜₑ(\vec{r}ⱼ^{D}, λ). Assumes that the fiber closest to the anomaly is the I-th source or J-th detector or both, so the anomaly is represented as Δμ(\vec{r}_{IJ}^{•}, λ). The anomaly causes a relative signal change of:

[symbol]

ψₕₑₜₑ(\vec{r}_{J}^{D}, λ)−ψₕₒₘₒ(\vec{r}_{J}^{D}, λ [symbol] /ψₕₒₘₒ(\vec{r}_{J}^{D}, λ)=W^{sens}(\vec{r}_{IJ}^{•}, \vec{r}_{J}^{D}, λ)·Δμ(\vec{r}_{IJ}^{•}, λ)

where W^{sens} is the sensitivity. For an idealized circular array, W^{sens} is uniform azimuthally and nearly uniform along the radial dimension. Therefore the relative signal change qualitatively maps Δμ(\vec{r}_{IJ}^{•}, λ). So a tissue heterogeneity index of

Δμ(\vec{r}ⱼ^{D}, λ)=Ψₕₑₜₑ(\vec{r}ⱼ^{D}, λ)/Ψₕₒₘₒ(\vec{r}ⱼ^{D}, λ)

can be assigned to the region under a detector fiber, and the tissue heterogeneity index of the region under a source fiber is interpolated by

Δμ(\vec{r}ᵢ^{S}, λ)= [symbol] ΣⱼΔμ(\vec{r}ⱼ^{D}, λ)W_{dist}^{(i,j)} [symbol] /ΣⱼW_{dist}^{(i,j)},

where the weight

W_{dist}^{(i,j)}= [symbol] exp(−|\vec{r}ᵢ^{S}−\vec{r}ⱼ^{D}| [symbol] /|\vec{r}ᵢ^{S}−\vec{r}ⱼ^{D}|²

scales the detector signals according to the distance of the source fiber. For spectral measurements, e.g., at λ₁=633 nm and λ₂=880 nm, it follows that

$$\frac{Δμ ( {{r}^{->}}_{i}^{S} , {λ}_{1} )}{Δμ ( {{r}^{->}}_{i}^{S} , {λ}_{2} )} = \frac{〈 {\sum }_{j} Δμ ( {{r}^{->}}_{j}^{D} , {λ}_{1} ) {W}_{dist}^{( i , j )} 〉 / {\sum }_{j} {W}_{dist}^{( i , j )}}{〈 {\sum }_{j} Δμ ( {{r}^{->}}_{j}^{D} , {λ}_{2} ) {W}_{dist}^{( i , j )} 〉 / {\sum }_{j} {W}_{dist}^{( i , j )}} \approx \frac{Δμ ( {{r}^{->}}_{j}^{D} , {λ}_{1} )}{Δμ ( {{r}^{->}}_{j}^{D} , {λ}_{2} )} ,$$

i.e., the ratio between the tissue heterogeneity indices at the two wavelengths directly represents the tissue spectral signature.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not to be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)−(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Still further, additional aspects of the instant invention may be found in one or more appendices attached hereto and/or filed herewith, the disclosures of which are incorporated herein by reference as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. An imaging device for imaging a tissue, comprising:
a. a light source;
b. a plurality of source fibers, each of said plurality of source fibers having a source fiber first end positionable to be in optical communication with the light source and an emitting end;
c. a plurality of detector fibers, each of said plurality of detector fibers having an imaging end positionable to be in optical communication with a light sensor and a detector end positioned to detect light from said plurality of source emitting ends that falls on the tissue; and,
d. a probe having an open upper end and an open terminus, wherein said upper end receives said plurality of source fibers and said plurality of detector fibers therein and guides said source and receiver fibers to said open terminus where said source and detector fibers emerge intermixed in a two-dimensional array of source fiber emitting ends and detector fiber ends within said probe terminus.

2. The imaging device according to claim 1, further comprising:
e. a display device; and,
f. a CPU in electronic communication with said light sensor and said display device, wherein said CPU is programmed to read light intensity information from said light sensor and display said light intensity information on said display device.

3. The imaging device according to claim 1, wherein said probe has a terminus of between 2 mm and 50 mm in diameter.

4. The imaging device according to claim 3, wherein there are between 50 and 350 source fibers and between 40 and 300 detector fibers.

5. The imaging device of claim 1 wherein each of said source fibers has a same diameter as each of said detector fibers.

6. The imaging device according to claim 3, wherein each source fiber and each detector fiber has a diameter of 750 μm.

7. The imaging device according to claim 1, wherein said light sort is an LED light source or a laser light source.

8. The imaging device according to claim 1, further comprising a diffuser situated between said light source and said source and said plurality of source fibers.

9. The imaging device according to claim 1, wherein said plurality of source fibers and said plurality of detector fibers emerge intermixed in a two-dimensional array of alternating source fiber emitting ends and detector fiber ends.

10. The imaging device according to claim 9, wherein said two-dimensional array of alternating source fiber emitting ends and detector fiber ends comprises alternating concentric circles of source fiber emitting ends and detector fiber ends.

11. An imaging device, comprising:
a. a first light source;
b. a plurality of first source fibers, each of said plurality of first source fibers having a first source fiber first end positionable to be in optical communication with the light source and a first source emitting end;
c. a second light source different from said first light source;
d. a plurality of second source fibers, each of said plurality of second source fibers having a second source fiber end positionable to be in optical communication with said second light source and a second source emitting end;
e. a plurality of detector fibers, each of said plurality of detector fibers having an imaging end positionable to be in optical communication with a light sensor and a detecting end; and,
f. a probe having an open upper end and an open terminus, wherein said upper end receives said plurality of first source fibers, said plurality of second source fibers, and said plurality of detector fibers therein and guides said first source fibers, said second source fibers, and said detector fibers to said open terminus where said first source fibers, said second source fibers, and said detector fibers emerge intermixed to form a two dimensional array of first source emitting ends, second source emitting ends, and detector ends within said probe terminus.

12. The imaging device according to claim 11, wherein said first light source emits light at a first single frequency and said second light source emits light at a second single frequency.

13. The imaging device according to claim 11, wherein said first light source is a broadband light source and said second light source emits light at a single frequency.

14. The imaging device according to claim 11, further comprising:
g. a display device; and,
h. a CPU in electronic communication with said light sensor and said display device, wherein said CPU is programmed to read light intensity information from said light sensor and display said light intensity information on said display device.

15. A method of imaging a tissue wherein is provided the apparatus of claim 1, comprising the steps of:
a. activing said light source;
b. directing said probe toward the tissue;
c. while said light source is activated collecting light information from each of said detector fibers imaging ends, thereby obtaining at least one light intensity value for each of said detector fibers;
d. associating each of said detector fiber ends with a position within said probe terminus; and,
e. using said position within said probe terminus associated with each of said detector fiber ends and said at least one light intensity value for each of said detector fibers to form a two dimensional image of said tissue.

16. A method of imaging a tissue, wherein is provided a probe and a plurality of source fibers and a plurality of detector fibers, each of said plurality of source fibers having a source end and an emitter end, and each of said detector fibers having a detector end and an imaging end, wherein said probe encases said source fibers and said detector fibers and terminates in plurality of intermixed said source ends and said detector ends that foam a two-dimensional array at an end of said probe, comprising the steps of:
a. activing said light source;
b. exposing each of said source ends to said light source;
c. directing said probe toward the tissue;
d. while said light source is activated collecting light from each of said imaging ends of said detector fibers, thereby obtaining at least one light intensity value for each of said detector fibers;
e. associating each of said detector fiber ends with a position within said probe terminus; and,
f. using said position within said probe terminus associated with each of said detector fiber ends and said at least one light intensity value for each of said detector fibers to form a two-dimensional image of said tissue.
